(19) United States
(12) Patent Application Publication
(10) Pub. No.: US 2013/0229620 A1
(43) Pub. Date: Sep. 5, 2013

(54) Enhanced Sensitivity Line Field Detection

(71) Applicants: Daniel X. Hammer, Bedford, MA (US); R. Daniel Ferguson, Melrose, MA (US); Mircea Mujat, Acton, MA (US); Nicusor V. Iftimia, Chelmsford, MA (US)

(72) Inventors: Daniel X. Hammer, Bedford, MA (US); R. Daniel Ferguson, Melrose, MA (US); Mircea Mujat, Acton, MA (US); Nicusor V. Iftimia, Chelmsford, MA (US)

(21) Appl. No.: 13/411,826

(22) Filed: Mar. 5, 2012

(51) Int. Cl. A61B 3/14

(52) U.S. Cl. 351/206; 351/246

(57) ABSTRACT

A retinal imaging device includes an optical system configured to (i) scan a portion of the retina of the eye with a line of light, (ii) descan reflected light from the scanned portion of the retina, and (iii) provide output light in a line focus configuration. The device includes a detection device including a linear array of asymmetric pixels having at least a 2:1 ratio of length to width, a detection device with multiple adjacent linear arrays, and/or a detection device using a time delay and integration (TDI) architecture.

### GOVERNMENT RIGHTS

The invention was made with government support under Department of Defense contract no. W81XWH-06-C-0397. The government has certain rights in the invention.

### FIELD OF THE INVENTION

The invention relates generally to enhanced line field retinal imaging, and more particularly, to a line scanning ophthalmoscope incorporating a detection device with asymmetric pixels, a detection device with multiple adjacent linear arrays, and/or a detection device using a time delay and integration (TDI) architecture.

### BACKGROUND

In vivo ophthalmic images are produced by back-reflected light from ocular tissue (e.g., retina, cornea, lens, etc.) and can overcome the problem of weak signals for applications such as detection of exogenous and endogenous fluorescence, molecular signatures, high-speed dynamics, multiphoton imaging, and others. In general, fundus imaging can be divided into three major device categories: flood illumination, line scanning or line field, and confocal. A fourth type, optical coherence tomography (OCT), uses an interferometric approach for light detection and is a fundamentally different technique. This application concerns new methodology for the detection of weak signals in a line scanning ophthalmic imager. Although many applications can benefit from the methodology presented, fluorescence detection is discussed first.

Endogenous fluorophores exist in the eye and have been used to characterize ocular health, provide early detection of retinal disease, and to quantify retinal constituents at the molecular level. Exogenous fluorescent dyes were first used in humans in the early 1960's and 1970's, and a few (e.g., fluorescein and indocyanine green (ICG)) have been approved for widespread use thereafter for the detection of retinal diseases. Dye angiography is used principally to detect either leakage from abnormal vessels, pooling of serous fluids and blood below the photoreceptors (edema, retinal detachments), or blockage of flow in the retinal and choroidal vessels leading to local ischemia.

Indocyanine green angiography (ICGA) and fluorescein angiography (FA) are usually used to identify specific areas of blood vessel leakage in the choriocapillaris and retinal vasculature. Fluorescein absorbs in the 485-490 nm wavelength range and emits at ˜530 nm, and can be useful for visualization of retinal vasculature in front of the highly pigmented retinal pigment epithelium (RPE) layer. Unbound sodium fluorescein is contained in the retinal vasculature by their tight junctions but diffuses through choriocapillaris vessels. ICG is excited and fluoresces at longer wavelengths in the near infrared (e.g., 800 and 835 nm), and these more deeply penetrating wavelengths are useful for visualization of choroidal circulation. ICG also has a higher molecular weight and binds nearly completely to plasma proteins. The dye leakage, and hence available imaging time, is longer.

Detection of weak fluorescence signals from the eye has been a challenge since the invention of the ophthalmoscope. Flash fundus photography uses a short duration white light flood illumination and synchronized full field detection to collect enough back-scattered light from the eye for imaging. While digital flash fundus photography is the clinical standard for angiography, it is difficult to configure for continuous imaging (e.g., video imaging) to visualize the entire sequence of dye infusion from early phase transit to late phase pooling.

Scanning laser ophthalmoscopy (SLO) is a confocal imaging technique that provides higher contrast imaging and depth sectioning via reduction of light scatter that arises from adjacent voxels. Because fluorescent signal is already confined to the retinal and choroidal vasculature, the depth sectioning capability of SLO's is less advantageous for exogenous dye angiography. For detection of autofluorescence (AF), though, SLO's provide an advantage because lenticular AF signals are automatically rejected by the confocal pinhole. Configuration of an SLO for AF or dye angiography usually involves a larger pinhole (with respect to the Airy disc), compared to that used for reflectance SLO images, thereby improving light sensitivity at the expense of scattered light rejection and depth sectioning.

Recently introduced clinical SLO imagers, such as the Heidelberg Spectralis, are multimodal systems that can collect optical coherence tomography (OCT), SLO, AF, FA, and ICGA images continuously. The fluorescent signal detection (AF, FA, ICGA) is usually performed with image averaging or at a relatively low frame rate compared to reflectance imaging (1-2 fps vs. 15-60 fps), and the Spectralis includes eye tracking for fluorescence image stabilization.

The line scanning ophthalmoscope (LSO) and line scanning laser ophthalmoscope (LSLO) are a quasi-confocal line field imaging techniques. LSO technology provides advantages over traditional SLO technology including only one moving part, simplified optics, and a compact footprint, even for dual detector applications such as stereoscopic visualization. The digital linear detector is programmable and usually easier to configure than the analog photomultiplier tube (PMT) or avalanche photodiode (APD) used in an SLO. The LSO can also attain higher frame rates and resolution than an SLO, because the image speed (e.g., line rate) is not constrained by moving hardware (for the SLO a spinning polygon or resonant scanner). This may be important for some research applications that involve dynamic events (retinal flow).

In both SLO and LSO, the illumination and detection optics determine the image resolution and confocality. The scanned point (in SLO) or line (in LSO) is de-scanned by the same optics back to the detector, setting the device magnification. The SLO point and LSO line are focused by the illumination optics to a diameter whose limit is ultimately determined by ocular aberrations. The confocality and resolution are determined by the size of the pinhole with respect to the Airy disc of the imaging optics. The LSO pixel, as opposed to the pinhole in an SLO, produces its confocality. Because of the fixed pixel size, the light collection properties cannot be controlled as easily as an SLO, which can be configured with adjustable or different-sized pinholes. In the axis adjacent to the line, the size is usually much smaller than an SLO pinhole (e.g., 14 μm vs. 100 μm). Thus, the LSO operates in a hyper-confocal regime with respect to the Airy disc. In the axis along the line, there is reduced confocality because light cross-talk from adjacent pixels occurs. Thus, the LSO is termed “line confocal” or “quasi-confocal,” though the contrast improvement and image appearance is typically similar between an SLO and LSO.

Line scanning angiography (LSA), which uses LSO at its core and includes software and instrumentation modifications to accomplish fluorescence angiography, has been demonstrated with fluorescein and indocyanine green dyes in humans. Existing LSA techniques do not collect extremely low light levels to enhance LSA for AF, FA, and ICGA.

### SUMMARY OF THE INVENTION

The invention features, in various embodiments, enhanced line field detection of signals (e.g., fluorescent signals) from the eye. One of three types of detections schemes can be used to enhance sensitivity. The detection schemes include using a detection device with asymmetric pixels, a detection device with multiple adjacent linear arrays, and/or a detection device using a time delay and integration (TDI) architecture and technique for binning pixels for improved sensitivity. Broadly, these detectors all share the capability of programmable control. Fluorescence detection components can be incorporated into a line scanning ophthalmoscope platform. Multiple image modalities (e.g., reflectance and fluorescence) can be merged for enhanced retinal disease visualization, and a therapeutic beam can be incorporated for disease treatment.

In one aspect, there is a retinal imaging device including an optical system configured to (i) scan a portion of a retina of an eye with a line of light, (ii) descan reflected light from the scanned portion of the retina, and (iii) provide output light in a line focus configuration. The apparatus includes a detection device configured to detect the output light and to image the portion of the eye. The detection device includes a linear array of asymmetric pixels having at least a 2:1 ratio of length to width.

In another aspect, there is a method for imaging a retina of an eye. The method includes scanning a line of light along a portion of the retina, descanning reflected light from the scanned portion of the retina, providing output light in a line focus configuration, and detecting a signal associated with an image of the portion of the retina scanned using a linear array of asymmetric pixels having at least a 2:1 ratio of length to width.

In still another aspect, there is an apparatus for imaging a retina of an eye. The apparatus includes means for scanning a line of light along a portion of the retina, means for descanning reflected light from the scanned portion of the retina, means for providing output light in a line focus configuration, and means for detecting a signal associated with an image of the portion of the retina scanned using a linear array of asymmetric pixels having at least a 2:1 ratio of length to width. The apparatus can include means for tracking a reference feature of the retina of the eye and means for controlling the position of the line of light relative to the reference feature to correct for motion of the eye.

In certain embodiments, the line of light is received along the linear array of asymmetric pixels. The asymmetric pixels can have a ratio from 2:1 to 10:1 of length to width.

In yet another aspect, there is a retinal imaging device including an optical system configured to (i) scan a portion of a retina of an eye with a line of light, (ii) descan reflected light from the scanned portion of the retina, and (iii) provide output light in a line focus configuration. The apparatus includes a detection device configured to detect the output light. The detection device includes multiple linear arrays of pixels. The apparatus includes a controller configured to sum charges from vertically adjacent pixels and to form an image of the portion of the retina scanned from the charges summed.

In another aspect, there is a method for imaging a retina of an eye. The method includes scanning a line of light along a portion of the retina, descanning reflected light from the scanned portion of the retina, providing output light in a line focus configuration, detecting a signal associated with an image of the portion of the retina scanned using multiple linear arrays of pixels, summing charges from vertically adjacent pixels, and forming the image the portion of the retina scanned from the charges summed.

In still another aspect, there is an apparatus for imaging a retina of an eye. The apparatus includes means for scanning a line of light along a portion of the retina, means for descanning reflected light from the scanned portion of the retina, means for providing output light in a line focus configuration, means for detecting a signal associated with an image of the portion of the retina scanned using multiple linear arrays of pixels, means for summing charges from vertically adjacent pixels, and means for forming the image the portion of the retina scanned from the charges summed. The apparatus can include means for tracking a reference feature of the retina of the eye and means for controlling the position of the line of light relative to the reference feature to correct for motion of the eye.

In certain embodiments, multiple horizontal rows of the multiple linear arrays are illuminated, and charges from vertically adjacent pixels of the multiple horizontal rows are summed. The image is created as if only a single linear array was illuminated. The detection device and/or the multiple linear arrays of pixels can include up to 100 (e.g., from 2 to 20) rows of pixels in a vertical axis.

In another aspect, there is a retinal imaging device including an optical system configured to (i) scan a portion of a retina of an eye with a line of light, (ii) descan reflected light from the scanned portion of the retina, and (iii) provide output light in a line focus configuration. A detection device is configured to detect the output light. The detection device includes multiple linear arrays of pixels. A controller is configured to synchronize scanning of the line of light on the retina and exposure on the multiple linear arrays of pixels to increase exposure time for each spatial region of the retina scanned.

In still another aspect, there is a method for imaging a retina of an eye. The method includes scanning a line of light along a portion of the retina, descanning reflected light from the scanned portion of the retina, providing output light in a line focus configuration, detecting a signal associated with an image of the portion of the retina scanned using multiple linear arrays of pixels, and synchronizing scanning of the line of light on the retina and exposure on the multiple linear arrays of pixels to increase exposure time for each spatial region of the retina scanned.

In yet another aspect, there is an apparatus for imaging a retina of an eye. The apparatus includes means for scanning a line of light along a portion of the retina, means for descanning reflected light from the scanned portion of the retina, means for providing output light in a line focus configuration, means for detecting a signal associated with an image of the portion of the retina scanned using multiple linear arrays of pixels, and means for synchronizing scanning of the line of light on the retina and exposure on the multiple linear arrays of pixels to increase exposure time for each spatial region of the retina scanned. The apparatus can include means for tracking a reference feature of the retina of the eye and means for controlling the position of the line of light relative to the reference feature to correct for motion of the eye.

In certain embodiments, the controller is configured to effect time delay integration to accumulate multiple exposures of each spatial region beam to increase exposure time. The controller can be configured (1) to cause (i) a first linear array of pixels to sample a first spatial region of the retina during a first time period, (ii) a second linear array of pixels to sample a second spatial region of the retina during a second time period, (iii) the first linear array of pixels to sample the first spatial region of the retina during a third time period, and (iv) the second linear array of pixels to sample the second spatial region of the retina during a fourth time period, (2) to sum first charge from the first linear array of pixels during the first time period and the third time period to form a first image of the first spatial region, and (3) to sum second charge from the second linear array of pixels during the second time period and the fourth time period to form a second image of the second spatial region. The detection device can include up to 100 (e.g., 2 to 64) rows of pixels.

In certain embodiments, the method includes sampling a first spatial region of the retina during a first time period using a first linear array of pixels, sampling a second spatial region of the retina during a second time period using a second linear array of pixels, sampling the first spatial region of the retina during a third time period using the first linear array of pixels, sampling the second spatial region of the retina during a fourth time period using the second linear array of pixels, forming a first image of the first spatial region by summing first charge from the first linear array of pixels during the first time period and the third time period, and forming a second image of the second spatial region by summing second charge from the second linear array of pixels during the second time period and the fourth time period.

In other examples, any of the aspects above, or any apparatus, system or device, or method, process or technique, described herein, can include one or more of the following features.

Fluorescence from endogenous or exogenous chromophores in the retina can be received (e.g., by the detection device). Reflectance from the retina of the eye can be received alone or in conjunction with the fluorescence.

In certain embodiments, a target feature in the image of the portion of the retina scanned can be identified, and a therapeutic beam can be delivered to the target feature to treat the eye.

The retinal imaging device can include a retinal tracking device for tracking a reference feature of the retina of the eye. The retinal tracking device can be configured to control the line of light's position relative to the reference feature to correct for motion of the eye.

The retinal tracking device can provide a signal to stabilize a source for the line of light relative to the reference feature and/or a signal to stabilize a source for a therapeutic beam relative to the reference feature. The signal can be the same signal or different signals. The line of light can share a common optical path with the therapeutic beam. The retinal tracking device can control the position of the line of light and the therapeutic beam relative to the reference feature.

Other aspects and advantages of the invention will become apparent from the following detailed description, taken in conjunction with the accompanying drawings, illustrating the principles of the invention by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 shows an illustrative embodiment of a retinal imaging device.

FIGS. 2A-2C show three detection schemes that can provide enhanced sensitivity line scanning imaging.

FIG. 3 shows a schematic diagram of an optical apparatus including a retinal tracking device for imaging a retina of an eye.

FIG. 4 shows a block diagram of a multimodal TSLO (mLSO system) that can combine retinal tracking, wide field confocal imaging, FA, IGA and a therapeutic treatment beam into a compact, slit-lamp-mounted platform.

FIG. 5 shows an optical layout for a retinal imaging system.

FIG. 6 shows an optical layout for a retinal imaging system including a therapeutic source.

FIG. 7 shows a comparison of fundus and fluorescence images from a human subject without retinal disease.

### DESCRIPTION OF THE INVENTION

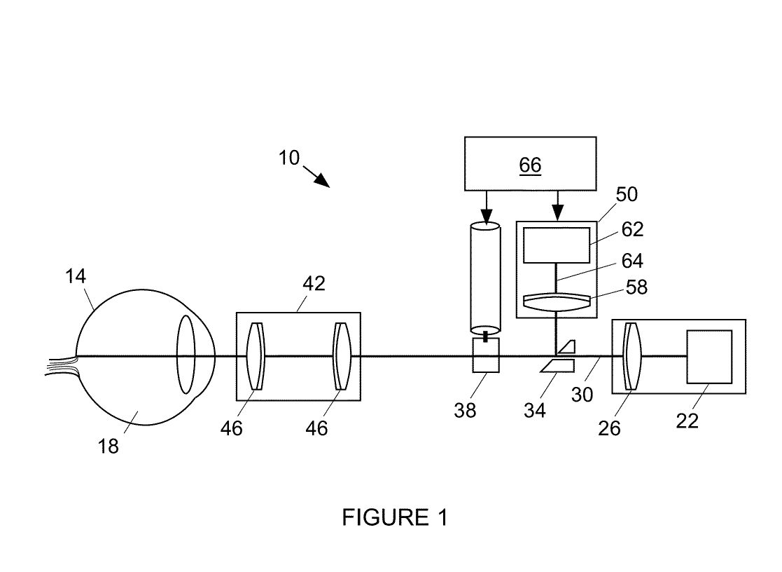

FIG. 1 shows an illustrative embodiment of a retinal imaging device 10 including and an optical system configured to (i) scan a portion of a retina 14 of an eye 18 with a line of light, (ii) descan reflected light from the scanned portion of the retina, and (iii) provide output light in a line focus configuration. The optical system can be a line scan ophthalmoscope (LSO), e.g., a line scan laser ophthalmoscope (LSLO) or a LSO using a diode or superluminescent diode (SLD). The optical system can provide an image having a wide field of view.

The optical system includes a source 22 and a lens system 26 to form the input beam 30 into a line of light. The optical system includes a beam separator 34 including an aperture through which the line of light passes and includes a galvanometer 38 to control the position of the imaging beam. The optical system includes an ocular interface 42 including one or more ophthalmic lens 46 for focusing the line of light in the retina.

Light returning from the eye 18 passes through the ocular interface 42 and is directed by the beam separator 34 to a detection system 50, including an objective lens 58 and a detection device 62 receiving light 64. A controller 66 can include a frame grabber and software to control the galvanometer 38 and the detection device 62.

The detection device 62 is configured to detect the output light and to image the portion of the eye. The detection device can include a linear array of asymmetric pixels (e.g., having at least a 2:1 ratio of length to width) and/or can include multiple linear arrays of pixels (e.g., square pixels). The controller 66 can be configured to sum charges from vertically adjacent pixels and to form an image of the portion of the retina scanned from the charges summed. In certain embodiments, the controller 66 is configured to synchronize scanning of the line of light on the retina and exposure on the multiple linear arrays of pixels to increase exposure time for each spatial region of the retina scanned.

The retinal imaging system 10 can include a retinal tracking device for tracking a reference feature of the retina of the eye. The retinal tracking device can control the position of the line of light relative to the reference feature to correct for motion of the eye. The retinal tracking device and the optical system can be referred to as a tracking line scanning ophthalmoscope (T-LSLO or TSLO).

In some embodiments, the system can include a port that can be used for delivery of near-diffraction-limited stimulus or therapeutic beams to the retina. The stimulus or therapeutic beam can require an external independent focus and can be collimated into the port.

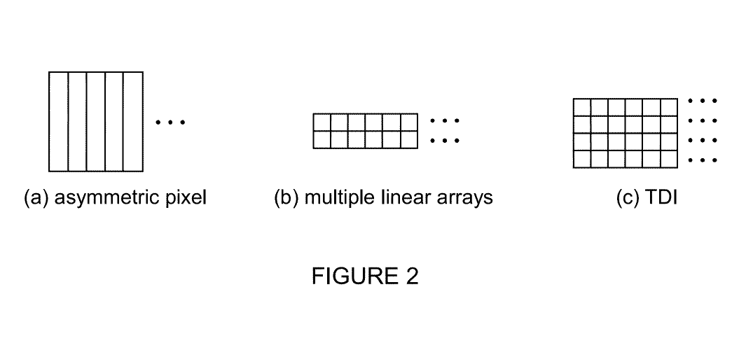

FIG. 2A-2C shows three detection schemes that can provide enhanced sensitivity line scanning imaging. FIG. 2A shows a linear array of asymmetric pixels. FIG. 2B shows multiple linear arrays of pixels. FIG. 2C shows multiple linear arrays of pixels used with a TDI methodology. Enhanced detector sensitivity is effected by using a detection device with detector elements having greater size or greater effective size.

Asymmetric pixels improve sensitivity without sacrificing lateral resolution in the axis along the detector. The asymmetric pixel can lead to a slight loss of confocality, degraded contrast, and reduced resolution adjacent to the detector. Detectors with asymmetric pixels are manufactured by several vendors (e.g., Basler and e2v). The asymmetric pixel can have any fixed ratio of horizontal to vertical dimensions (e.g., a 2:1 ratio or about 14×28 μm pixel size). Other ratios can be used, e.g., up to 10:1.

A process utilizing a detector with asymmetric pixels can track a reference feature of the retina of the eye, scan a line of light along a portion of the retina, control the position of the line of light relative to the reference feature to correct for motion of the eye, descan reflected light from the scanned portion of the retina, provide output light in a line focus configuration, and detect a signal associated with an image of the portion of the retina scanned using a linear array of asymmetric pixels. The line of light is received along the linear array of asymmetric pixels (e.g., along a horizontal axis if the pixels extend in a vertical axis).

While asymmetric pixels can solve the problem of low sensitivity with larger pixels for greater light collection, multiple adjacent lines of pixels can add a degree of programmability that enhances lateral resolution and allows real-time configuration of the confocal aperture. These detectors have two or more adjacent lines with square pixels that can be summed (e.g., binned) in a number of different ways for improved light sensitivity. Simultaneous vertical binning, where all the vertically adjacent pixels are summed to create a single line, is similar, if not equivalent, to an asymmetric pixel whose ratio is equal to the number of lines. Time delayed integration, where one line of pixels is summed with the pixels from the previous line, is similar to TDI.

A process utilizing a detector with multiple linear arrays of pixels can track a reference feature of the retina of the eye, scan a line of light along a portion of the retina, control the position of the line of light relative to the reference feature to correct for motion of the eye, descan reflected light from the scanned portion of the retina, provide output light in a line focus configuration, detect a signal associated with an image of the portion of the retina scanned using multiple linear arrays of pixels, sum charges from vertically adjacent pixels, and form the image the portion of the retina scanned from the charges summed. In certain embodiments, multiple horizontal rows of the multiple linear arrays are illuminated, and charges from vertically adjacent pixels of the multiple horizontal rows are summed. The image is created as if only a single linear array was illuminated. The detection device and/or the multiple linear arrays of pixels can include up to 100 (e.g., from 2 to 20) rows of pixels in a vertical axis.

Several manufactures produce linear detectors with multiple lines (e.g., the Basler Sprint). Currently, TDI sensors have a large number of adjacent lines (e.g., 64) with square pixels that are summed in successive line periods. These detectors allow the largest degree of programmability and control over the confocal aperture for line scanning angiography imaging. The advantage to the TDI approach is that a single spatial region can be sampled with high resolution for a long integration time without loss of overall image speed.

TDI is typically used in industrial applications where the target is conveyed across the synchronized sensor. One location on the moving sample is imaged on a line and the same location is imaged on an adjacent line one line period later. The image of that location is thus built up from several summed (or binned) lines. The number of lines that are binned in TDI is completely programmable.

In an LSO, the image of the scanned illuminated line on the retina is de-scanned by the galvanometer back to the detector and so there is no moving image. However, adjacent lines of the linear detector sample adjacent spatial regions just like a moving target. Summing across several periods thus samples the same spatial region for a longer integration time without increasing the frame rate. The number of TDI lines used to improve sensitivity in LSO/LSA can be determined, in part, by the width of the illumination line, since the fluorescent molecules have very short decay times (nanoseconds) and are excited only when illuminated. The illumination line width can be configured. TDI for LSO/LSA corresponds to collecting the cumulative distribution function of the Airy disc function.

An advantage of TDI is that the lateral resolution and confocality are preserved in the axis adjacent to the line. Whereas asymmetric pixels collect light from the spatial region subtended by the size of the vertical pixel, TDI, which uses square pixels, provides finer sampling of that spatial region, to the limit of the LSO confocal technique.

For example, a process utilizing a detector with multiple linear arrays of pixels can track a reference feature of the retina of the eye, scan a line of light along a portion of the retina, control the position of the line of light relative to the reference feature to correct for motion of the eye, descan reflected light from the scanned portion of the retina, provide output light in a line focus configuration, detect a signal associated with an image of the portion of the retina scanned using multiple linear arrays of pixels, and synchronize scanning of the line of light on the retina and exposure on the multiple linear arrays of pixels to increase exposure time for each spatial region of the retina scanned.

In certain embodiments, the controller is configured to effect time delay integration to accumulate multiple exposures of each spatial region beam to increase exposure time. The controller can be configured (1) to cause (i) a first linear array of pixels to sample a first spatial region of the retina during a first time period, (ii) a second linear array of pixels to sample a second spatial region of the retina during a second time period, (iii) the first linear array of pixels to sample the first spatial region of the retina during a third time period, and (iv) the second linear array of pixels to sample the second spatial region of the retina during a fourth time period, (2) to sum first charge from the first linear array of pixels during the first time period and the third time period to form a first image of the first spatial region, and (3) to sum second charge from the second linear array of pixels during the second time period and the fourth time period to form a second image of the second spatial region. The detection device can include up to 100 (e.g., 2 to 64) rows of pixels.

In certain embodiments, the method includes sampling a first spatial region of the retina during a first time period using a first linear array of pixels, sampling a second spatial region of the retina during a second time period using a second linear array of pixels, sampling the first spatial region of the retina during a third time period using the first linear array of pixels, sampling the second spatial region of the retina during a fourth time period using the second linear array of pixels, forming a first image of the first spatial region by summing first charge from the first linear array of pixels during the first time period and the third time period, and forming a second image of the second spatial region by summing second charge from the second linear array of pixels during the second time period and the fourth time period.

The detection device can be configured to receive and/or collect fluorescence from endogenous or exogenous chromophores in the retina. Reflectance from the retina of the eye can be received alone or in conjunction with the fluorescence.

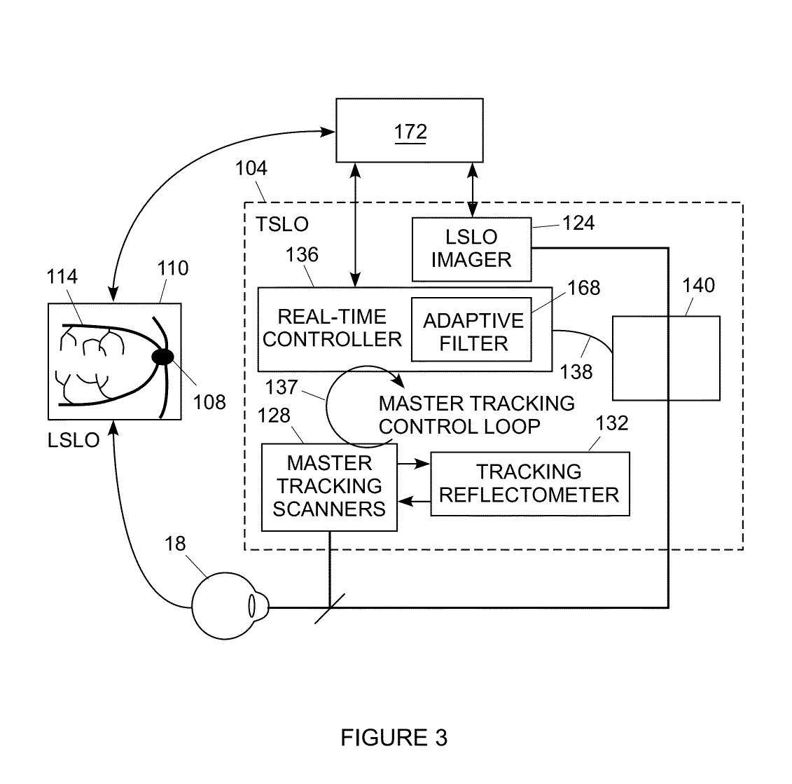

Referring to FIG. 3, the retinal imaging system 104 can track a reference feature 108 shown in a wide-field image 110 of the retina of an eye 18. The reference feature 108 can be the optic nerve head. Other features of the eye that can be used as the reference feature 108 include features of the fundus, such as blood vessel junctions, scleral crescents, foveal pits, and regions of hypopigmentation in a subject with a diseased eye. Other features of the eye shown in the image 110 include blood vessels 114. The retinal imaging system 104 includes an LSLO 124 to provide the wide-field image 110, although a LSO can be used instead. The retinal imaging system 104 includes a first tracking device 128 for controlling the position of a tracking beam relative to the reference feature 108 and for controlling the position of an imaging beam associated with the LSLO 124. A reflectometer 132 can provide an output signal with a phase corresponding to a phase of the reflected tracking beam, and a signal processor 136 can compare the phase of the reflectometer output signal to the phases of the oscillatory motion in the first and second direction, generating the “master” tracking control loop 137.

The retinal imaging system 104 can stabilize high magnification images by driving two galvanometers 140 placed at appropriate conjugates within the path of the retinal imaging system 104 in a “master-slave” configuration. The input to the master control loop 137 is x-y error signals generated from the low power track beam (˜100 μW measured at the cornea, 1060-nm laser diode, LD) dithered on a retinal feature and detected from a confocal reflectometer 132. The input to the slave control loop 138 is the scaled position signals from the master galvanometers 128. The slave tracking galvanometers 140 can be placed at conjugates to the center of rotation of the eye. This allows line-of-sight tracking (i.e., simultaneous tracking of the pupil and retina from rotational eye motion) because the mirrors pivot about the true axis of rotation of the eye 18. Targets can be tracked anywhere within a greater than about the 40 degree LSLO field.

The tracking functions can be performed by a single set of stacked electronics boards. A single printed circuit board can also be used. Communication between the tracking board(s) and host computer is accomplished via a USB interface. Control parameters are passed to and reflectance, position, and error signals are received from the tracking board(s).

A blink detection and track re-lock algorithm can be used. The control and processing electronics include a field programmable gated array (FPGA) chip that performs digital lock-in amplification and other pre-processing steps, a digital signal processor (DSP) that performs two proportional-integral-derivative (PID) control loops (for master and slave systems), and analog-to-digital and digital-to analog converters (ADC and DACs) to receive reflectometer signals and drive galvanometers. The DSP has a loop rate of about 62.5 kHz for a closed loop bandwidth well in excess of 1 kHz. The tracking beam wavelength can be in the 800-900-nm range, or can be 1060 nm to accommodate a superluminescent diode (SLD). Other tracking beam wavelengths can be used as well. The resonant scanner—and dither frequency—can be 8 kHz or 16 kHz, although other frequencies can be used.

An exemplary LSLO system is described in U.S. Pat. No. 6,758,564, the disclosure of which is herein incorporated by reference in its entirety. An exemplary tracking system is described in U.S. Pat. No. 5,797,941, the disclosure of which is herein incorporated by reference in its entirety.

The tracking galvanometers 140 can include a first input and a second input. The first input can accept a first direction control signal from the signal processor 136, and the second input can accept a second direction control signal from the signal processor 136, causing the imaging beam to track relative to the reference feature 108. In some embodiments, an adaptive filter 168 filters the direction control signals sent from the signal processor 136 to the tracking galvanometers 140.

For example, the master tracker can use a reflectometer error signal generated from phase-sensitive detection of the dithered tracking beam in a control loop (e.g., elements 128, 132, and 136). For slave tracking operation, a separate control loop can be run that uses the positions of the master galvanometers as the input. The adaptive filter 168 can provide calibration and filtering to the slave galvanometer 140. Mirrors can be placed at both the pupil and center-of-rotation conjugates. The offsets of at least one or all of the scanners, including the resonant scanner, can be adjusted. Therefore, a scanner can be moved to any angle. Direction control signals can drive the offset of the resonant scanner, causing the imaging beam to track translationaly relative to the reference feature.

Controller 172 can receive information from the detection device of the LSLO imager 124 and can include a display device to render image 110. Controller 172 can be or can include a processor to sum charges from vertically adjacent pixels and to form the image 110 of the portion of the retina scanned from the charges summed. Controller 172 can be or can include a processor to synchronize scanning of the line of light on the retina and exposure on the multiple linear arrays of pixels to increase exposure time for each spatial region of the retina scanned. Controller can be in communication with controller 136 to effect tracking and imaging of the eye 18.

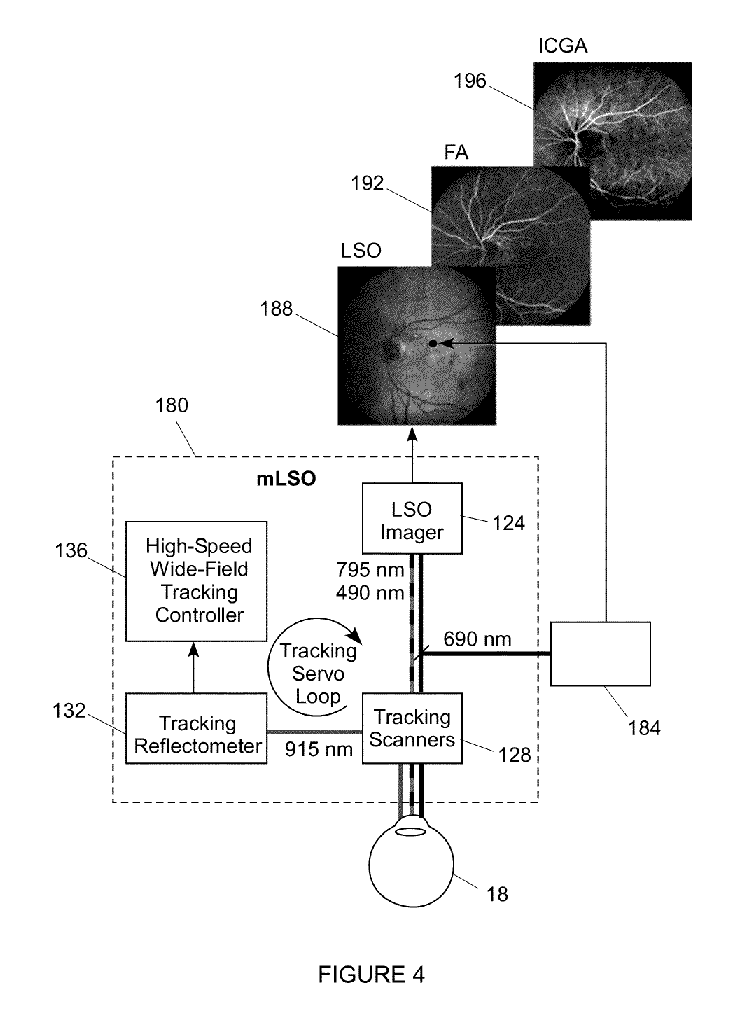

FIG. 4 shows a block diagram of a multimodal TSLO (mLSO system) 180 that can combine retinal tracking, wide field confocal imaging, FA, IGA and a therapeutic treatment beam into a compact, slit-lamp-mounted platform. A target feature in the image of the portion of the retina scanned can be identified, and a therapeutic beam can be delivered to the target feature to treat the eye. The confocal imager can used a detection device having enhanced sensitivity. The mSLO system 180 includes a confocal LSO imager 124 configured for angiography (490-nm and 795-nm excitation), a retinal tracker (915-nm) 128, 132, 136, 137 and a therapeutic source 184 (e.g., a photodynamic therapy (PDT) treatment laser (690 nm beam)). The treatment beam can be delivered through tracking scanners to fixed retinal coordinates. The system can provide high contrast images of the fundus to monitor the PDT laser spot and eye position data during any imaging or therapeutic procedure. An advantage of the mLSO is the ability to merge the reflectance 188, FA 192, and ICGA 196 images to obtain a clearer picture of ocular health.

The therapeutic source 184 can deliver radiation to treat or stimulate the retina, or a portion thereof, of the eye 18. The beam can be near-diffraction-limited. For example, the beam can be a therapeutic beam delivered to the eye 18 to treat a target feature of the retina. The target feature can be a retinal pigment epithelial (RPE) cell, a feeder vessel, a drusen, a small tumor, a microaneurysm, or an epiretinal membrane. The retinal imaging system can provide a high resolution, wide field image of the target feature, and the beam can be delivered to precisely target and treat the target feature.

In some embodiments, the therapeutic source 184 can deliver a near-diffraction-limited stimulus beam that can be used for vision studies. The beam can be delivered to the eye 18 so that measurements of the eye or the retina can be recorded. In some embodiments, the tracking galvanometer 140 can control the position of the beam and the imaging beam relative to a target feature.

The radiation beam can be a coherent beam of light (e.g., generated by a laser) or an incoherent beam of light (e.g., generated by a pulsed light source or a light emitting diode). In some embodiments, the pulse duration of the source is less than about 1 μsec. For example, the beam can have a pulse duration in the femto, pico, or nano second time regime. The laser can be an ultra short pulsed laser, which can confine thermal injury to the target feature and avoid unwanted damage to surrounding tissue.

In some embodiments, the radiation is collimated into a port or coupled into the system using an optic positioned behind the LSO 124. The radiation can require an external independent focus. The retinal tracking device can provide a signal to stabilize a source for the line of light relative to the reference feature and/or a signal to stabilize a source for a therapeutic beam relative to the reference feature. The signal can be the same signal or different signals. The line of light can share a common optical path with the therapeutic beam. The retinal tracking device can control the position of the line of light and the therapeutic beam relative to the reference feature. A diffraction-limited spot can be delivered to confined, precise, and fixed coordinates on the retina, a feature that typically can not be performed without retinal tracking Using an ultra-short pulsed device can further confine the damage.

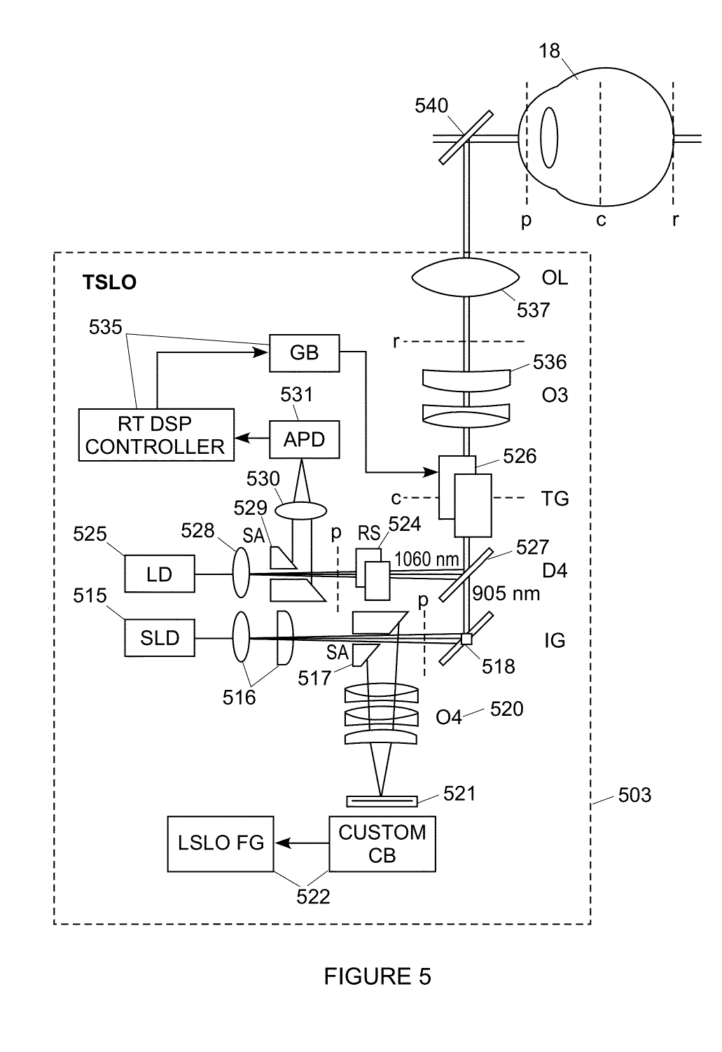

FIG. 5 shows an optical layout for another embodiment of a retinal imaging system 503, which includes a first module 503 to track a reference feature of an eye 18. The retinal imaging system 503 can be a line scanning ophthalmoscope for wide-field images combined with a retinal tracking system. The LSO includes a source 515 for an imaging beam, a system of lenses 516 (including a cylindrical lens) to form the point source to a line of light, a beam separator 517 with an aperture to separate the scanned beam from the descanned beam, a galvanometer driven mirror 518 to scan the imaging beam along a portion of the eye 18, and an objective lens 520 to focus radiation returning from the eye to a detection device 521 to acquire a wide-field image of a portion of the eye. The detector 521 is in electrical communication with a framegrabber 522 for acquiring a plurality of images of the eye 18.

The detection device 521 can include a linear array of asymmetric pixels having at least a 2:1 ratio of length to width and/or can include multiple linear arrays of pixels (e.g., square pixels).

The retinal imaging system 503 includes a dithering device 524 (e.g., a resonant scanner) to dither a tracking beam provided by source 525 in a first and second direction with an oscillatory motion on the eye. In some embodiments, the dithering device 524 can be a resonant scanner. The retinal imaging system 503 also has a tracking device 526, such as a galvanometer, to control the position of the tracking beam and the imaging beam associated with the LSLO. The tracking galvanometer 526 can be placed at the conjugate associated with the center of rotation of the eye. The resonant scanner can be placed at or near the retinal conjugate. The beam paths of the retinal tracker and the LSLO can be combined with a beam splitter 527 (e.g., a pellicle or dichroic beam splitter).

The source 525 for the tracking beam passes through a lens 528 and through the aperture of beam separator 529. A lens 530 focuses the return beam to an avalanche photo detector 531 in electrical communication with a signal processor or controller 535 that can send direction control signals to the first tracking device 526. A photomultiplier tube can be used in place of an avalanche photo detector. Lens 536 and 537 focus the imaging beam and the tracking beam to the eye 18. Optic 540 can redirect the beam to the eye. In certain embodiments, optic 540 is not used and the eye is aligned along the axis of the beam focused by ophthalmoscopic lens 537.

Source 515 can be a laser, a diode or a superluminescent diode. For example, source 515 can be a superluminescent diode operating at 905 nm or 760 or a laser diode at 795 nm.

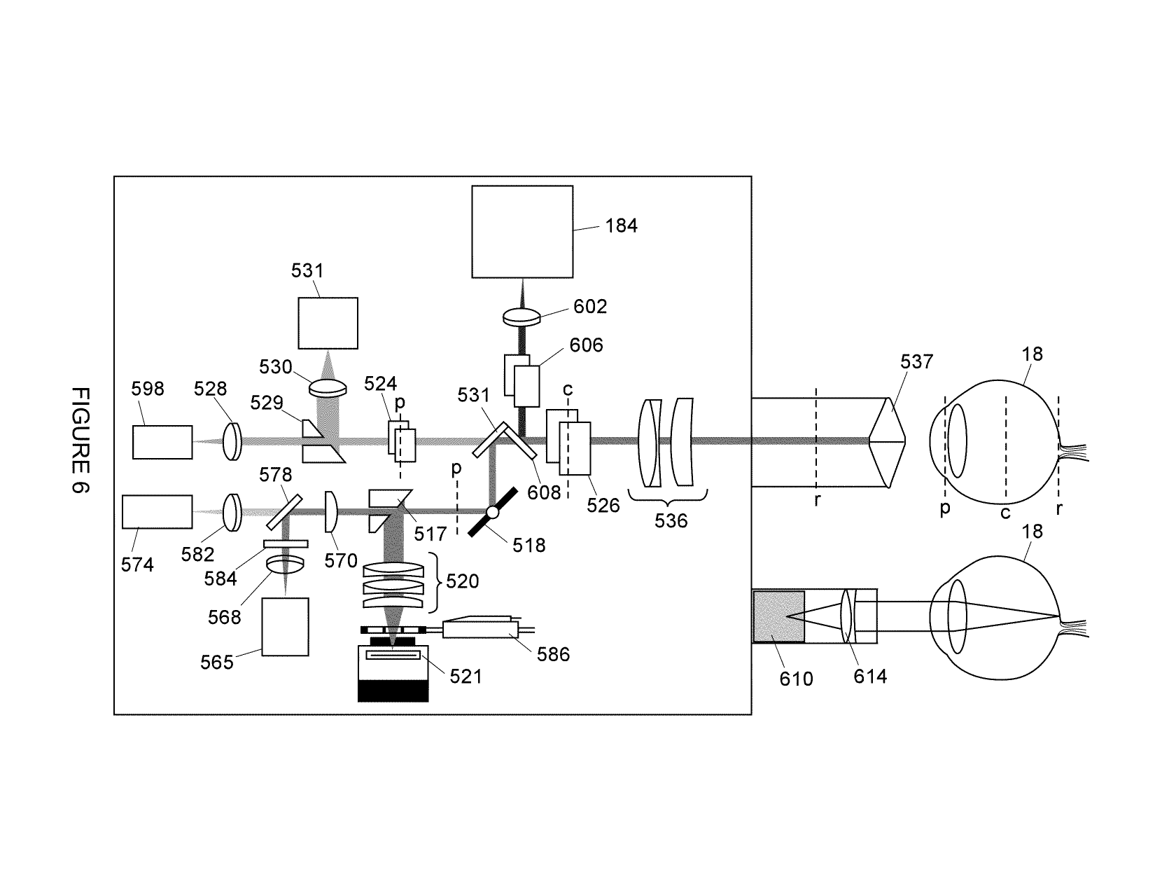

FIG. 6 shows a mLSO retinal imaging and treatment system 560 including a tracking scanning laser ophthalmoscope (TSLO). The mLSO has been configured with sources, filters, and dichroic beamsplitters to collect near-infrared confocal reflectance images, ICG angiograms, and fluorescein angiograms. The LSO hardware includes a source 565, a lens 568, a cylindrical lens 570, a beam separator 517 with an aperture, a single scanning galvanometer 518, and a detector 521. The imaging optics include a detector objective 520 and a scan objective 536. The objectives can include one or more lens. A front ophthalmoscopic lens 537 is used to direct radiation to the eye 18. The instrument can be configured with several different ophthalmoscopic lenses (e.g., Volk 40D, 66D, 78D) to achieve a variety of retinal field sizes.

Source 565 is used for ICG angiography. Source 574 is used for fluorescein excitation. Dichroic beamsplitter 578 can be used to combine the beam paths of the radiation from sources 565 and 574. Source 565 can be a 795 nm laser diode or a 760 nm superluminescent diode (SLD) (Exalos Inc). Source 574 can be a compact visible (490-nm with an output power of ˜25 mW, e.g., Cobolt Calypso laser) laser, including coupling optics (e.g., filters, fiber-couplers and lens 582). An excitation filter 584 (e.g., 751-798 nm) prevents illumination light from reflecting back into the detector in the ICG emission band. An illumination port can be added to the optical head to couple the fluorescein excitation laser beam into the optical beam path. A computer-controlled, motorized filter stage 586 is positioned before detector 521 to switch between reflectance, ICGA, and FA modes for interleaved operation and display.

Beamsplitter 578 can be a low-pass (˜600 nm cutoff) dichroic beamsplitter 578 to direct a 490-nm laser source used for FA into the optical path of the LSLO. Beamsplitter 590 can be a high-pass (˜900 nm cutoff) dichroic beamsplitter to combine the tracking and imaging beam paths. Beamsplitter 594 can direct the therapeutic laser 184 into the optical path of the imaging beam. Beamsplitter 594 can be a custom band-pass filter available from Omega Optical Inc. with performance at both 490 and 795 nm.

The retinal tracking system includes source 598, lens 528, beam separator 529, resonant scanners 524, lens 530 and detector 531. Source 598 can be a 915-nm superluminescent diode (SLD) for retinal tracking Tracking galvanometers 526 correct for motion of the eye.

Therapeutic source 184 can be a commercially available PDT laser (e.g., Visulas 690s and Visulink PDT/U manufactured by Carl Zeiss Meditec Inc.). The therapeutic source 184 can have an output wavelength of 690 nm. The source is designed to be mounted on several different manufacturers' slit lamps. The PDT laser can include a control box, an optical port, and a foot switch. The controller has a touch screen to allow the user to change parameters such as light dose and exposure time. The therapeutic beam path includes lens 602 and galvanometers 606. Therapeutic source 184 is coupled into the optical path of the eye using a beamsplitter 608 (e.g., a dichroic or pellicle beamsplitter).

The filter stage 586 is designed to rapidly switch between emission filters (EF) for reflectance, ICGA, and FA using a Quickshaft linear actuator (Faulhaber). The actuator has a top speed of 1.9 m/s and uses Hall sensors to achieve a resolution of 6 μm, a repeatability of 40 μm, and a precision of 120 μm over the positioning range. Three filter slots house emission filters for ICGA and FA and Schott glass for confocal reflectance LSO imaging. The FA emission filter passes visible light from 507 to 562 nm and the ICGA emission filter passes NIR light from 819 to 863 nm. The filters can be made to the same optical thickness to prevent path length differences that can affect image focus when switching filters. The linear actuator is driven by commands from the controller with a small motion controller board integrated into the instrumentation box. In addition to switching emission filters, an FA illumination source shutter assembly can be mounted to the actuator so that when the filter stage is in any position other than FA, it will block the 490-nm laser beam from entering the eye.

The mLSO 560 includes an integrated LED-array fixation target 610 and lens 614. The fixation can introduce eye position errors but the fixation target is only intended to coarsely position the subject's gaze direction to lock and re-lock tracking on particular targets.

System electronics and instrumentation can be contained in a single instrumentation box. The electronics can be built around two custom printed circuit boards, a motherboard and field programmable gated array (FPGA)-based tracker controller board, and can include several commercial OEM driver electronics boards. The primary function of the tracker controller board is to independently run the feedback loop between the detector and galvanometers in real time.

The motherboard can have hardware to interface with OEM driver boards for the galvanometers (Cambridge Technology), resonant scanners (EOPC Inc.), and laser diodes and SLD sources (Exalos and QPhotonics). The motherboard also includes integrated LD/SLD thermo-electric coolers and drivers, integrated custom silicon detector electronics (for the tracking reflectometer), power regulation electronics, and header slots to mount and communicate with the custom retinal tracker control board. The tracker control electronics include two boards: a Xilinx Virtex-4 FPGA mini-module board and an analog front-end (AFE) that includes a 6-channel 250-kHz analog-to-digital converter (ADC), a 4-channel, 100-kHz digital-to-analog converter (DAC), digital input/output (DUO) lines, and serial communication with the host PC. The detector signal can be sampled at 208 kHz and the eye position data is transferred to PC at a decimated rate of 1 kHz.

In a clinical study, subjects were asked to gaze at the illuminated light on the LED array (spacing ˜2.5 deg.) during a patterned illumination sequence while tracking was active. The performance of the automatic blink/re-lock algorithm was also verified.

The clinical study was designed to perform complementary imaging on patients undergoing standard fundus camera angiography to verify mLSO performance. The mLSO system was set up in the clinical space immediately adjacent to a TopCon Fundus Photography System (model #TRC50X). Initial clinical testing was limited to verification and optimization of the angiography imaging modes alone. In the subject without retinal disease and the subject with CSC, FA and ICGA sequences were obtained during the initial dye-infusion stage. In all other patients, the subjects were first imaged on the TopCon system during dye injection and then moved over to the mLSO after a few minutes. This somewhat limited the visualization of early stage dye infusion, when the dye concentration is largest. However, this procedure did not hinder resolution of late stage leakage.

The mLSO was configured to acquire FA and ICGA images at 1 frame per second (fps). The power at the cornea was ˜0.6 mW and ˜1 mW for the individual FA (490 nm) and ICGA (795 nm) sources, which were not used simultaneously. Because the 490-nm source is extremely bright to the patient, the mLSO imager was configured to modulate the source by collecting three frames at 490 nm followed by seven LSO frames for every 10 frames acquired. The NIR LSO beam power was reduced to <50-100 μW for the longer integration time that accompanied the lower frame rate (the LSO is typically run at 15-60 fps). The software was configured to automatically switch between appropriate bandpass filters, trigger the ICGA shutter, and change to image brightness scaling to accommodate the different FA and LSO reflectance modes during acquisition of this sequence.

Because the mLSO and the TopCon always imaged an eye at different time-points with respect to the dye infusion, a direct comparison between the two devices was not possible. However, subjects were able to move between instruments to get a general idea of the appearance of early and late phase angiograms on both instruments.

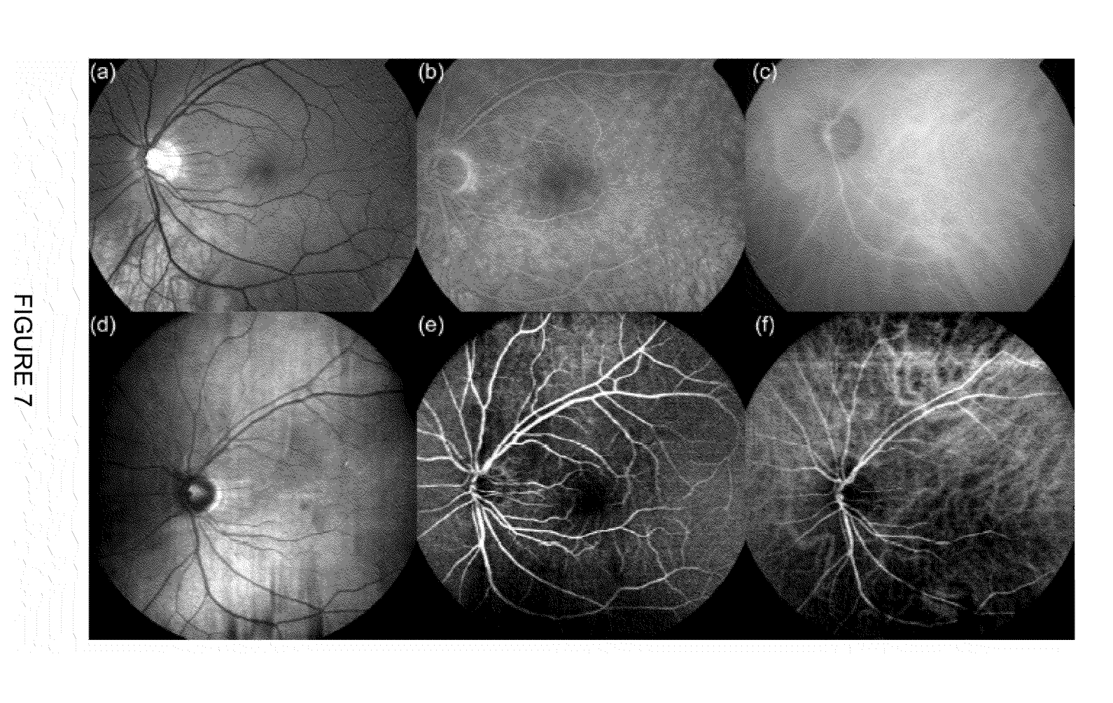

FIG. 7 shows a comparison of fundus and fluorescence images from a human subject without retinal disease. Top row images (a)-(c) are images from a TopCon fundus photography system. Bottom row images (d)-(f) are images from a retinal imaging system (e.g., a mSLO) of the technology. (a) Red-free image. (b) Late phase FA image [04:06 after injection of dye]. (c) Late phase ICGA image. (d) LSO confocal reflectance image. (e) Early phase FA image [00:46]. (f) Early phase ICGA image [00:48]. The field of view is about 50 degrees for both imagers. The mLSO FA image shows good resolution of all the retinal vasculature. As a consequence of the mLSO confocality, the choroidal vessels in the FA image are not as visible as in the TopCon. However, the mLSO ICGA image shows good contrast of the choroidal vessels.

The above-described techniques can be implemented in digital electronic circuitry, or in computer hardware, firmware, software, or in combinations of them. The implementation can be as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Method steps can be performed by one or more programmable processors executing a computer program to perform functions of the technology by operating on input data and generating output. Method steps can also be performed by, and apparatus can be implemented as, special purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), or an ASIC (application-specific integrated circuit), or the like. Subroutines can refer to portions of the stored computer program and/or the processor, and/or the special circuitry that implement one or more functions.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

The terms “module” and “function,” as used herein, mean, but are not limited to, a software or hardware component which performs certain tasks. A module may advantageously be configured to reside on addressable storage medium and configured to execute on one or more processors. A module may be fully or partially implemented with a general purpose integrated circuit (IC), DSP, FPGA or ASIC. Thus, a module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules may be combined into fewer components and modules or further separated into additional components and modules. Additionally, the components and modules may advantageously be implemented on many different platforms, including computers, computer servers, data communications infrastructure equipment such as application-enabled switches or routers, or telecommunications infrastructure equipment, such as public or private telephone switches or private branch exchanges (PBX). In any of these cases, implementation may be achieved either by writing applications that are native to the chosen platform, or by interfacing the platform to one or more external application engines.

To provide for interaction with a user, the above described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The above described techniques can be implemented in a distributed computing system that includes a back-end component, e.g., as a data server, and/or a middleware component, e.g., an application server, and/or a front-end component, e.g., a client computer having a graphical user interface and/or a Web browser through which a user can interact with an example implementation, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet, and include both wired and wireless networks. Communication networks can also all or a portion of the PSTN, for example, a portion owned by a specific carrier.

The computing system can include clients and servers. A client and server are generally remote from each other and typically interact through a communication network. The relationship of client and server arises by virtue of computer programs running on the respective computers and having a client-server relationship to each other.

While the invention has been particularly shown and described with reference to specific illustrative embodiments, it should be understood that various changes in form and detail may be made without departing from the spirit and scope of the invention.

What is claimed is:

1. A retinal imaging device comprising:
an optical system configured to (i) scan a portion of a retina of an eye with a line of light, (ii) descan reflected light from the scanned portion of the retina, and (iii) provide output light in a line focus configuration; and
a detection device configured to detect the output light and to image the portion of the eye, the detection device including a linear array of asymmetric pixels having at least a 2:1 ratio of length to width.

2. The retinal imaging device of claim 1 wherein the detection device is configured to receive the line of light along the linear array of asymmetric pixels.

3. The retinal imaging device of claim 1 wherein the asymmetric pixels have a ratio from 2:1 to 10:1 of length to width.

4. The retinal imaging device of claim 1 wherein the detection device is configured to receive fluorescence from endogenous or exogenous chromophores in the retina.

5. The retinal imaging device of claim 1 wherein the detection device is configured to receive fluorescence from endogenous or exogenous chromophores in the retina and to receive reflectance from the retina of the eye.

6. The retinal imaging device of claim 1 further comprising a retinal tracking device for tracking a reference feature of the retina of the eye, the retinal tracking device configured to control the line of light's position relative to the reference feature to correct for motion of the eye.

7. The retinal imaging device of claim 6 wherein the retinal tracking device is configured to provides a signal to stabilize a source for the line of light relative to the reference feature.

8. The retinal imaging device of claim 6 wherein the retinal tracking device is configured to provide a signal to stabilize a source for a therapeutic beam relative to the reference feature.

9. The retinal imaging device of claim 8 wherein the line of light shares a common optical path with the therapeutic beam, the retinal tracking device controls the line of light's position and the therapeutic beam's position relative to the reference feature.

10. A method for imaging a retina of an eye, comprising:
scanning a line of light along a portion of the retina;
descanning reflected light from the scanned portion of the retina;
providing output light in a line focus configuration; and
detecting a signal associated with an image of the portion of the retina scanned using a linear array of asymmetric pixels having at least a 2:1 ratio of length to width.

11. The method of claim 10 further comprising receiving the line of light along the linear array of asymmetric pixels.

12. The method of claim 10 wherein the linear array of asymmetric pixels have a ratio from 2:1 to 10:1 of length to width.

13. The method of claim 10 further comprising receiving fluorescence from endogenous or exogenous chromophores in the retina.

14. The method of claim 10 further comprising receiving fluorescence from endogenous or exogenous chromophores in the retina and receiving reflectance from the retina of the eye.

15. The method of claim 10 further comprising:
tracking a reference feature of the retina of the eye; and
controlling the line of light's position relative to the reference feature to correct for motion of the eye.

16. The method of claim 10 further comprising:
identifying a target feature in the image of the portion of the retina scanned; and
delivering a therapeutic beam to the target feature to treat the eye.

17. A retinal imaging device comprising:
an optical system configured to (i) scan a portion of a retina of an eye with a line of light, (ii) descan reflected light from the scanned portion of the retina, and (iii) provide output light in a line focus configuration;
a detection device configured to detect the output light, the detection device including multiple linear arrays of pixels; and
a controller configured to sum charges from vertically adjacent pixels and to form an image of the portion of the retina scanned from the charges summed.

18. The retinal imaging device of claim 17 wherein the detection device is configured to receive the line of light on multiple horizontal rows of the detection device, and the controller is configured to sum charges from vertically adjacent pixels of the multiple horizontal rows and create the image as if only a single linear array was illuminated.

19. The retinal imaging device of claim 17 wherein the detection device includes from 2 to 20 rows of pixels in a vertical axis.

20. The retinal imaging device of claim 17 wherein the detection device is configured to receive fluorescence from endogenous or exogenous chromophores in the retina.

21. The retinal imaging device of claim 17 wherein the detection device is configured to receive fluorescence from endogenous or exogenous chromophores in the retina and to receive reflectance from the retina of the eye.

22. The retinal imaging device of claim 17 further comprising a retinal tracking device for tracking a reference feature of the retina of the eye, the retinal tracking device configured to control the line of light's position relative to the reference feature to correct for motion of the eye.

23. The retinal imaging device of claim 22 wherein the retinal tracking device is configured to provide a signal to stabilize a source for the line of light relative to the reference feature.

24. The retinal imaging device of claim 22 wherein the retinal tracking device is configured to provide a signal to stabilize a source for a therapeutic beam relative to the reference feature.

25. The retinal imaging device of claim 24 wherein the line of light shares a common optical path with the therapeutic beam, the retinal tracking device controls the line of light's position and the therapeutic beam's position relative to the reference feature.

26. A method for imaging a retina of an eye, comprising:
scanning a line of light along a portion of the retina;
descanning reflected light from the scanned portion of the retina;
providing output light in a line focus configuration;
detecting a signal associated with an image of the portion of the retina scanned using multiple linear arrays of pixels;
summing charges from vertically adjacent pixels; and
forming the image the portion of the retina scanned from the charges summed.

27. The method of claim 26 further comprising:
illuminating multiple horizontal rows of the multiple linear arrays;
summing charges from vertically adjacent pixels of the multiple horizontal rows; and
creating the image as if only a single linear array was illuminated.

28. The method of claim 26 wherein the multiple linear arrays of pixels includes from 2 to 20 rows of pixels in a vertical axis.

29. The method of claim 26 further comprising receiving fluorescence from endogenous or exogenous chromophores in the retina.

30. The method of claim 26 further comprising receiving fluorescence from endogenous or exogenous chromophores in the retina and receiving reflectance from the retina of the eye.

31. The method of claim 26 further comprising:
tracking a reference feature of the retina of the eye; and
controlling the line of light's position relative to the reference feature to correct for motion of the eye.

32. The method of claim 26 further comprising:
identifying a target feature in the image of the portion of the retina scanned; and
delivering a therapeutic beam to the target feature to treat the eye.

33. A retinal imaging device comprising:
an optical system configured to (i) scan a portion of a retina of an eye with a line of light, (ii) descan reflected light from the scanned portion of the retina, and (iii) provide output light in a line focus configuration;
a detection device configured to detect the output light, the detection device including multiple linear arrays of pixels; and
a controller configured to synchronize scanning of the line of light on the retina and exposure on the multiple linear arrays of pixels to increase exposure time for each spatial region of the retina scanned.

34. The retinal imaging device of claim 33 wherein the controller is configured to effect time delay integration to accumulate multiple exposures of each spatial region beam to increase exposure time.

35. The retinal imaging device of claim 33 wherein the controller is configured to:
cause (i) a first linear array of pixels to sample a first spatial region of the retina during a first time period, (ii) a second linear array of pixels to sample a second spatial region of the retina during a second time period, (iii) the first linear array of pixels to sample the first spatial region of the retina during a third time period, and (iv) the second linear array of pixels to sample the second spatial region of the retina during a fourth time period;
sum first charge from the first linear array of pixels during the first time period and the third time period to form a first image of the first spatial region; and
sum second charge from the second linear array of pixels during the second time period and the fourth time period to form a second image of the second spatial region.

36. The retinal imaging device of claim 33 wherein the detection device includes from 2 to 64 rows of pixels.

37. The retinal imaging device of claim 33 wherein the detection device is configured to receive fluorescence from endogenous or exogenous chromophores in the retina.

38. The retinal imaging device of claim 33 wherein the detection device is configured to receive fluorescence from endogenous or exogenous chromophores in the retina and to receive reflectance from the retina of the eye.

39. The retinal imaging device of claim 33 further comprising a retinal tracking device for tracking a reference feature of the retina of the eye, the retinal tracking device configured to control the line of light's position relative to the reference feature to correct for motion of the eye.

40. The apparatus of claim 39 wherein the retinal tracking device is configured to provide a signal to stabilize a source for the line of light relative to the reference feature.

41. The apparatus of claim 39 wherein the retinal tracking device is configured to provide a signal to stabilize a source for a therapeutic beam relative to the reference feature.

42. The apparatus of claim 41 wherein the line of light shares a common optical path with the therapeutic beam, the retinal tracking device controls the line of light's position and the therapeutic beam's position relative to the reference feature.

43. A method for imaging a retina of an eye, comprising:
scanning a line of light along a portion of the retina;
descanning reflected light from the scanned portion of the retina;
providing output light in a line focus configuration;
detecting a signal associated with an image of the portion of the retina scanned using multiple linear arrays of pixels; and
synchronizing scanning of the line of light on the retina and exposure on the multiple linear arrays of pixels to increase exposure time for each spatial region of the retina scanned.

44. The method of claim 43 further comprising effecting time delay integration to accumulate multiple exposures of each spatial region beam to increase the exposure time.

45. The method of claim 43 further comprising:
sampling a first spatial region of the retina during a first time period using a first linear array of pixels;
sampling a second spatial region of the retina during a second time period using a second linear array of pixels;
sampling the first spatial region of the retina during a third time period using the first linear array of pixels;
sampling the second spatial region of the retina during a fourth time period using the second linear array of pixels;
forming a first image of the first spatial region by summing first charge from the first linear array of pixels during the first time period and the third time period; and
forming a second image of the second spatial region by summing second charge from the second linear array of pixels during the second time period and the fourth time period.

46. The method of claim 43 wherein the multiple linear arrays of pixels includes from 2 to 64 rows of pixels.

47. The method of claim 43 further comprising receiving fluorescence from endogenous or exogenous chromophores in the retina.

48. The method of claim 43 further comprising receiving fluorescence from endogenous or exogenous chromophores in the retina and receiving reflectance from the retina of the eye.

49. The method of claim 43 further comprising:
tracking a reference feature of the retina of the eye; and
controlling the line of light's position relative to the reference feature to correct for motion of the eye.

50. The method of claim 43 further comprising:
identifying a target feature in the image of the portion of the retina scanned; and
delivering a therapeutic beam to the target feature to treat the eye.
